(19) United States
(12) Patent Application Publication
(10) Pub. No.: US 2018/0110414 A1
(43) Pub. Date: Apr. 26, 2018

(54) PHOTODYNAMIC DIAGNOSTIC DEVICE AND PHOTODYNAMIC DIAGNOSTIC METHOD

(71) Applicant: SONY CORPORATION, Tokyo (JP)

(72) Inventors: Koichiro KISHIMA, Kanagawa (JP); Hiroshi MAEDA, Kanagawa (JP); Takuya KISHIMOTO, Kanagawa (JP); Takashi YAMAGUCHI, Kanagawa (JP); Kazuki IKESHITA, Saitama (JP)

(73) Assignee: SONY CORPORATION, Tokyo (JP)

(21) Appl. No.: 15/559,495

(22) PCT Filed: Feb. 25, 2016
(86) PCT No.: PCT/JP2016/055683

§ 371 Date: Sep. 19, 2017

(30) Foreign Application Priority Data
Apr. 27, 2015 (JP) 2015-090041

(51) Int. Cl. A61B 5/00; A61B 5/055; A61B 6/03; A61B 8/08; G06T 11/60; G06T 7/00; G06T 7/30

(52) U.S. Cl. CPC A61B 5/0071; A61B 5/0035; A61B 5/0091; A61B 5/055; A61B 6/032; A61B 8/0825; G06T 2207/30096; G06T 7/0012; G06T 7/30; A61B 2576/02; G06T 2207/10064; G06T 2207/30196; G06T 11/60

(57) ABSTRACT

The location of a tumor can be more easily and accurately recognized in the photodynamic diagnosis in which: a fluorescence image is produced by radiating excitation light having a specific wavelength from an excitation light source and capturing an image of fluorescence from a photosensitizer excited by the excitation light by a fluorescence imaging device; and an integrated image is produced by integrating the fluorescence image with a first image representing a positional relation of at least a part of a human body.

### TECHNICAL FIELD

The present disclosure relates to a photodynamic diagnostic device and a photodynamic diagnostic method.

### BACKGROUND ART

In general, tumor cells forming malignant tumors are juvenile, and porphyrin-based substances inside the cells are easily bound to lipoproteins and slowly excreted to the outside of the cells. On the basis of such characteristics, administration of porphyrin-based drugs to the body enables to create a difference in the concentration of the drugs between normal cells and tumor cells by utilizing a difference in the excretion rate between the normal cells and the tumor cells. This led to the development of drugs with tumor selectivity and eventually a photosensitizer capable of visualizing the difference in the concentration of the drugs by a photochemical reaction in which the drugs were excited by externally applied light energy to obtain fluorescence. Utilizing such a photosensitizer enables to visualize the presence of the tumor cells with fluorescence. The diagnosis using a combination of the photosensitizer and light is referred to as Photo Dynamic Diagnosis (PDD) and used in a wide range of clinical areas. A device for PDD has been also developed (see e.g., Patent Literature 1 below).

### CITATION LIST

### Patent Literature

Patent Literature 1: JP 2014-25774A

### DISCLOSURE OF INVENTION

### Technical Problem

The PDD mentioned above may be performed during the excision surgery of the tumors to determine the presence of the malignant tumors that are not completely removed. However, in order to obtain a fluorescence image captured by the PDD (hereinafter, also referred to as a “PDD image”), it is important to lower a quantity of illumination light from an external light source such as a shadowless lamp as much as possible. The PDD image captured in this manner is a simple image, in which a part where the fluorescence is generated is present on a dark background (e.g., a background entirely in black). Thus, although the presence of the malignant tumors can be determined by referring to the PDD image, it is extremely difficult to specify the location of the malignant tumors in an actual operation field only using the PDD image.

Thus, there has been a demand for a method that makes it possible to recognize the location of the malignant tumors more easily and accurately during the excision surgery of the malignant tumors.

Accordingly, the present disclosure proposes a photodynamic diagnostic device and photodynamic diagnostic method that make it possible to recognize the location of the malignant tumors more easily and accurately in view of the aforementioned circumstances.

### Solution to Problem

According to the present disclosure, there is provided a photodynamic diagnostic device including: an imaging unit including an excitation light source that radiates excitation light having a specific wavelength and a fluorescence imaging device that captures an image of fluorescence from a photosensitizer excited by the excitation light to produce a fluorescence image; and an arithmetic processing unit including an image processing unit that applies predetermined image processing to the fluorescence image. The image processing unit integrates a first image representing a positional relation of at least a part of a human body into the fluorescence image to produce an integrated image.

Further, according to the present disclosure, there is provided a photodynamic diagnostic method including: producing a fluorescence image by radiating excitation light having a specific wavelength from an excitation light source and capturing an image of fluorescence from a photosensitizer excited by the excitation light by a fluorescence imaging device; and producing an integrated image by integrating a first image representing a positional relation of at least a part of a human body into the produced fluorescence image.

According to the present disclosure, an imaging unit captures an image of fluorescence generated from a photosensitizer excited by excitation light to produce a fluorescence image, and an arithmetic processing unit integrates a first image representing a positional relation of at least a part of a human body into the produced fluorescence image to produce an integrated image.

### Advantageous Effects of Invention

According to the present disclosure described above, it becomes possible to recognize the location of malignant tumors more easily and accurately.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram illustrating a photodynamic diagnostic device according to a first embodiment of the present disclosure.

FIG. 2 is a block diagram schematically illustrating an example of overall configurations of the photodynamic diagnostic device according to the first embodiment.

FIG. 3 is an explanatory diagram schematically illustrating an example of configurations of an imaging unit of the photodynamic diagnostic device according to the first embodiment.

FIG. 4 is an explanatory diagram illustrating photosensitizers and their excitation wavelengths.

FIG. 5 is an explanatory diagram schematically illustrating another configuration example of the imaging unit according to the first embodiment.

FIG. 6 is a block diagram schematically illustrating an example of configurations of an arithmetic processing unit of the photodynamic diagnostic device according to the first embodiment.

FIG. 7 is a block diagram schematically illustrating an example of configurations of an image processing unit included in the arithmetic processing unit according to the first embodiment.

FIG. 8 is an explanatory diagram illustrating production processes of a display image performed in the image processing unit according to the first embodiment.

FIG. 9 is an explanatory diagram illustrating production processes of a display image performed in the image processing unit according to the first embodiment.

FIG. 10 is an explanatory diagram illustrating production processes of a display image performed in the image processing unit according to the first embodiment.

FIG. 11 is a flowchart illustrating an example of processes performed in a photodynamic diagnostic method according to the first embodiment.

FIG. 12 is a block diagram illustrating an example of hardware configurations of the arithmetic processing unit according to the embodiment of the present disclosure.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

Note that the explanation is given in the following order.

1. Aim

2. First Embodiment

2. 1. Overall configuration of photodynamic diagnostic device
2. 2. Configuration of imaging unit
2. 3. Configuration of image processing unit
2. 4. Photodynamic diagnostic method

3. Hardware configuration of image processing unit

### (Aim)

Prior to describing a photodynamic diagnostic device and photodynamic diagnostic method according to an embodiment of the present disclosure, the aim of the photodynamic diagnostic device and photodynamic diagnostic method according to the embodiment of the present disclosure is described in more detail using the breast cancer as an example of tumors developed in a human body.

With the development of modem technologies, giving a chemotherapy to shrink or eliminate tumors prior to the excision surgery (Neo Adjuvant chemotherapy: NAC) is more commonly adopted as a treatment strategy of the breast cancer. In recent years, treatment results of the breast cancer and prognosis of patients have been improved with the advent of molecularly-targeted drugs such as Herceptin (registered trademark). Under such circumstances, the NAC is developed as a therapeutic method in which drugs effective to the breast cancer are administered prior to the excision surgery. As a result, the tumors become undetectable before the surgery in an image diagnosis using a technique such as a CT in some cases. Although a region corresponding to the cancer (a cancer region) seems to be eliminated in the image diagnosis using a technique such as a CT, quite a few cancer cells may remain, thus the excision surgery is commonly performed after the NAC.

Following the introduction of the NAC, a therapeutic protocol from diagnosis to surgery for the breast cancer mainly includes a cancer screening diagnosis by a mammography, a diagnosis by a needle biopsy, a neo adjuvant chemotherapy (NAC), and excision surgery in this order. Further, during the excision surgery, an excision target area on the breast of a patient is commonly marked by a marker pen on the basis of diagnostic images such as a mammographic image, a CT image, an MRI image, and an ultrasonic image.

On the other hand, it has become clear that the shrinkage of the cancer region by the NAC depends on types of the breast cancer and, in some type of the breast cancer, the tumors shrink as a whole while leaving the scattered cancer regions in the surroundings. Further, as the cancer region disappears, fibrosis and inflammation in its surroundings also disappear, which causes a change in the overall shape of the breast and makes the region occupied by the tumors before the NAC unclear. These factors increase a risk of insufficient excision of the tumors (so-called a risk of positive surgical margins) in the excision surgery.

Further, unlike the endoscopic operation, a physician as an operator commonly performs a surgical operation while viewing the operation field in the conventional excision surgery of the breast cancer. This also makes it difficult to find the remaining cancer regions scattered by the NAC in the surroundings. Further, improvement of the prognosis of the patients promotes minimizing a surgical excision region in a cosmetic point of view in order to further enhance QOL of the patients, thus causing concern in increasing the risk of positive surgical margins.

Further, the risk of positive surgical margins described above is not limited to the breast cancer, but also exists in other malignant tumors.

Thus, the additional use of the PDD during the surgery as described above is effective to further reduce the risk of positive surgical margins described above. However, the PDD image obtained by the PDD only shows the location of the cancer cells in which the photosensitizers used in PDD accumulate. Thus, when the PDD image alone is used, the operator can easily recognize the presence of the cancer cells, but hardly recognize the location of the cancer cells in the operation field.

The present inventors conducted intensive studies regarding the above-mentioned points to seek a technique that makes it possible to more easily and accurately recognize the locations of malignant tumors. As a result, the present inventors came up with an idea of integrating the PDD image obtained by the PDD into a first image different from the PDD image as described in detail below, thereby completing a technique based on the present disclosure described in detail below.

### First Embodiment

### <Overall Configuration of Photodynamic Diagnostic Device>

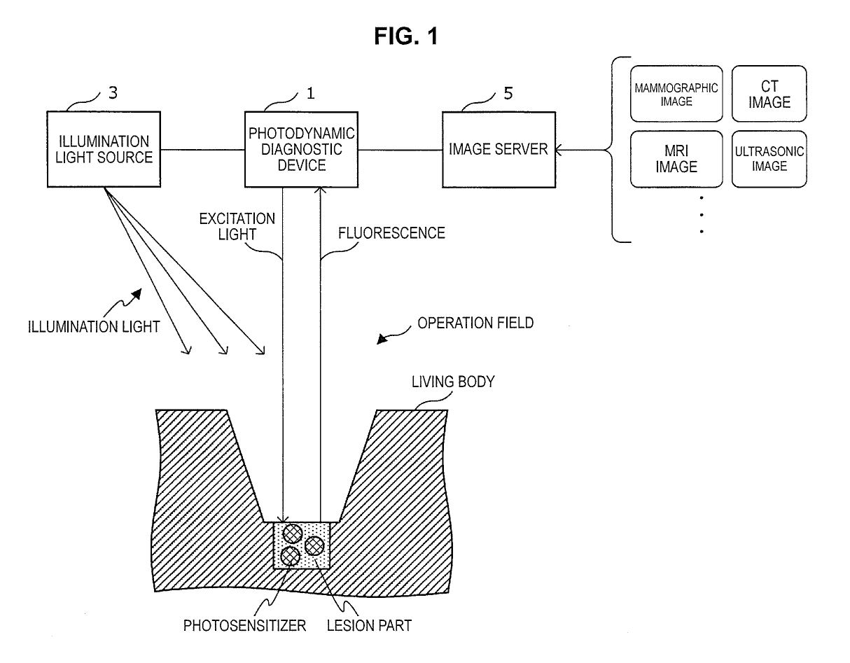

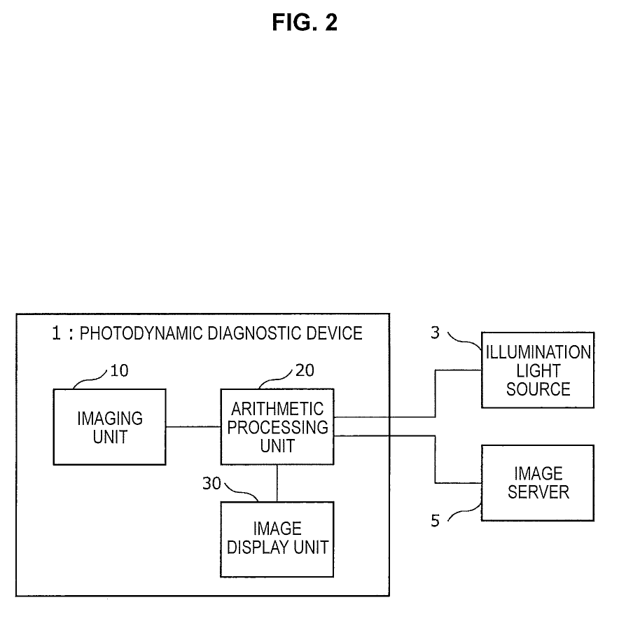

Next, the overall configuration of a photodynamic diagnostic device according to a first embodiment of the present disclosure will be described in detail with reference to FIG. 1 and FIG. 2. FIG. 1 and FIG. 2 show explanatory diagrams schematically illustrating an example of the overall configurations of the photodynamic diagnostic device according to the present embodiment.

As schematically shown in FIG. 1, a photodynamic diagnostic device 1 according to the present embodiment radiates excitation light having a specific wavelength to a part of a human body to which photosensitizers administered in advance are highly likely to be accumulated (e.g., a lesion part of malignant tumors such as cancer) and captures an image of fluorescence from the photosensitizers excited by the excitation light. The photosensitizers are selectively accumulated in tumors cells forming malignant tumors such as cancer, thereby making it possible to determine the presence of the tumors cells by the presence of the fluorescence, that is, making it possible to perform so-called PDD.

Further, as schematically shown in FIG. 1, the photodynamic diagnostic device 1 according to the present embodiment performs the PDD cooperatively with an illumination light source 3 for radiating illumination light to an operation field of the excision surgery, an image server 5 for storing data of various medical images, and the like.

In this configuration, the illumination light source 3 radiates the illumination light belonging to a visible light band to the operation field and no particular limitation is imposed on a detailed structure of the illumination light source 3. The illumination light source 3 may be a publicly known light source such as a shadowless lamp already installed in an operation room or the like, or a light source separately installed from the shadowless lamp or the like. The illumination light source 3 may include an own illumination light control mechanism or be controlled by the photodynamic diagnostic device 1 according to the present embodiment for radiating the illumination light.

Note that FIG. 1 shows the case in which the illumination light source 3 that radiates the illumination light belonging to a visible light band is installed separately from the photodynamic diagnostic device 1, however, the photodynamic diagnostic device 1 according to the present embodiment may further include an illumination light source that radiates illumination light belonging to a visible light band. Including the own illumination light source in the photodynamic diagnostic device 1 can omit an operation to turn on and off the shadowless lamp.

The image server 5 stores the data of the various medical images and is configured to be accessible from the photodynamic diagnostic device 1 via a publicly known network such as an internet and a local area network. The image server 5 stores the various diagnostic images that show the locations of malignant tumors such as cancer. Such diagnostic images include a fluoroscopic image fluoroscopically visualizing at least a part of a human body and a sectional image capturing a cross section of at least a part of a human body. Examples of the fluoroscopic image and the sectional image include a mammographic image, a CT image, an MRI image, and an ultrasonic image, however, the fluoroscopic image and the sectional image referred in the present embodiment are not limited to the above-mentioned images and also include any image data used for diagnosis at a medical scene.

The photodynamic diagnostic device 1 can access to the image server 5 at any time to utilize the various diagnostic images stored in the image server 5 in image processing described below.

The photodynamic diagnostic device 1 that performs the PDD cooperatively with the various devices described above mainly includes an imaging unit 10, an arithmetic processing unit 20, and an image display unit 30, as schematically shown in FIG. 2.

The imaging unit 10 radiates exciting light having a specific wavelength to at least a part of a human body to which the photosensitizers are administered in advance and captures an image of the fluorescence from the photosensitizers excited by the exciting light to produce a fluorescence image. Further, the imaging unit 10 may include a mechanism for further radiating the illumination light belonging to a visible light band in addition to the exciting light having a specific wavelength. Detailed configuration of the imaging unit 10 will be described again below.

The arithmetic processing unit 20 applies predetermined image processing to the fluorescence image produced by the imaging unit 10 to produce image data that allow a user of the photodynamic diagnostic device 1 (i.e., an operator of the excision surgery of the malignant tumors) to obtain the fluorescence image in a format to be easily understood. During this process, the arithmetic processing unit 20 can acquire various image data from the image server 5 arranged outside the photodynamic diagnostic device 1 and supply the various image data to the image processing performed in the arithmetic processing unit 20.

Further, the arithmetic processing unit 20 functions as a control unit that controls various imaging processes performed in the imaging unit 10 and thus can control various light sources and imaging devices and various optical apparatuses included in the imaging unit 10. Further, the arithmetic processing unit 20 can also control the illumination light radiated from the illumination light source 3.

Detailed configuration of the arithmetic processing unit 20 will be also described again below.

The image display unit 30 presents various image data produced by applying the image processing to the fluorescence image in the arithmetic processing unit 20 to a user of the photodynamic diagnostic device 1. The image display unit 30 is configured from one or more various displays and the like. Display of various images on the image display unit 30 is controlled by the arithmetic processing unit 20. The image display unit 30 presents the fluorescence image processed to be easily understood to a user of the photodynamic diagnostic device 1. This allows the user of the photodynamic diagnostic device 1 to recognize the presence of the fluorescence from the photosensitizers (i.e., the presence of the remaining malignant cells) and, if the malignant cells remain, to easily recognize the location of the remaining malignant cells.

In the foregoing, the overall configuration of the photodynamic diagnostic device 1 according to the present embodiment has been described in detail with reference to FIG. 1 and FIG. 2.

### <Configuration of Imaging Unit 10>

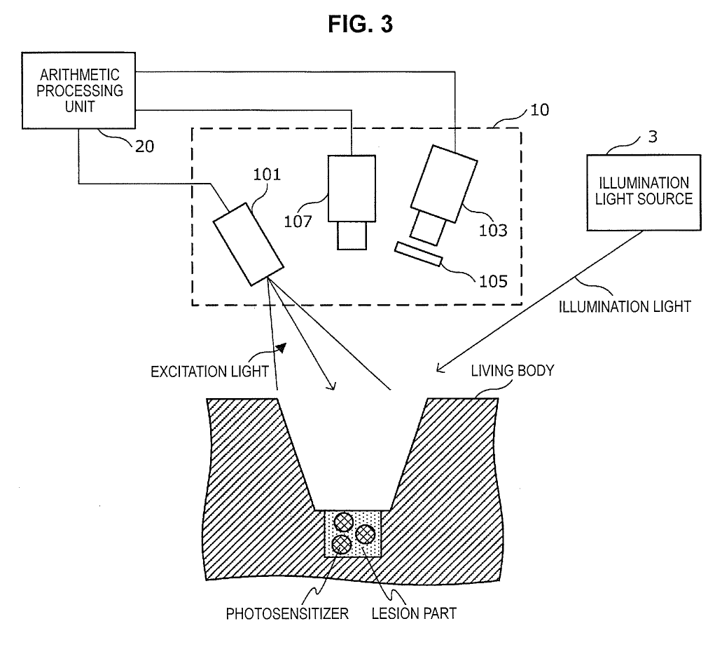

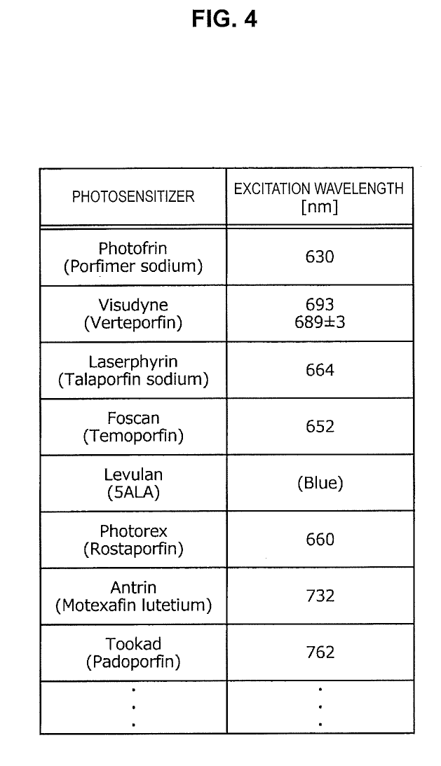

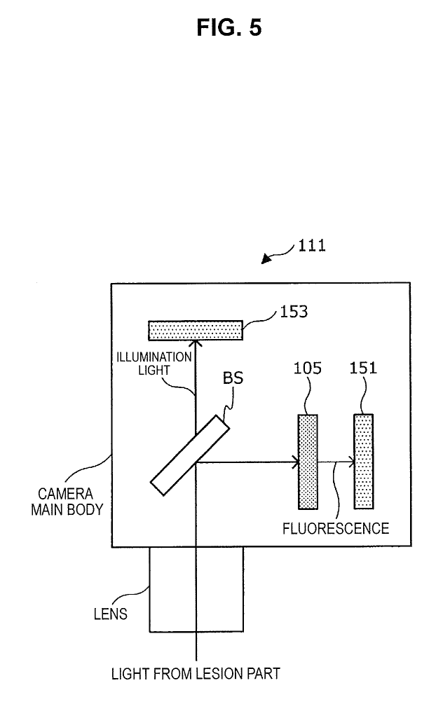

Next, the configuration of the imaging unit 10 provided in the photodynamic diagnostic device 1 according to the present embodiment will be described in detail with reference to FIG. 3 to FIG. 5. FIG. 3 shows an explanatory diagram schematically illustrating an example of configurations of the imaging unit of the photodynamic diagnostic device according to the present embodiment. FIG. 4 shows an explanatory diagram illustrating photosensitizers and their excitation wavelengths. FIG. 5 shows an explanatory diagram schematically illustrating another configuration example of the imaging unit according to the present embodiment.

The imaging unit 10 according to the present embodiment includes at least an excitation light source 101 and a fluorescence imaging device 103, as schematically shown in FIG. 3.

The excitation light source 101 radiates excitation light having a specific wavelength to at least a part of a human body including a lesion part where the photosensitizers are accumulated (i.e., malignant tumors such as cancer). The wavelength of the excitation light radiated from the excitation light source 101 is not particularly limited, and any wavelengths capable of exciting the photosensitizers accumulated in advance in the lesion part may be selected.

FIG. 4 shows combinations of examples of photosensitizers and their corresponding excitation wavelengths. Each photosensitizer is excited by a specific excitation wavelength according to its chemical structure. For example, Photofrin (registered trademark) representing one example of the photosensitizers is excited by the excitation light having a wavelength of 630 nm to emit fluorescence of a specific wavelength. Similarly, a photosensitizer called Visudyne (registered trademark) is excited by the excitation light having a wavelength of 693 nm or the excitation light having a wavelength of 689 nm±3 nm to emit fluorescence of a specific wavelength, and a photosensitizer called Laserphyrin (registered trademark) is excited by the excitation light having a wavelength of 664 nm to emit fluorescence of a specific wavelength. A photosensitizer called Foscan (registered trademark) is excited by the excitation light having a wavelength of 652 nm to emit fluorescence of a specific wavelength and a photosensitizer called Levulan (registered trademark) is excited by blue light to emit fluorescence of a specific wavelength. A photosensitizer called Photorex (registered trademark) is excited by the excitation light having a wavelength of 660 nm to emit fluorescence of a specific wavelength, a photosensitizer called Antrin (registered trademark) is excited by the excitation light having a wavelength of 732 nm to emit fluorescence of a specific wavelength, and a photosensitizer called Tookad (registered trademark) is excited by the excitation light having a wavelength of 762 nm to emit fluorescence of a specific wavelength.

Note that the photosensitizers and their excitation wavelengths shown in FIG. 4 are mentioned for example purposes only and not intended to limit the photosensitizers usable in the photodynamic diagnostic device 1 according to the present embodiment.

The wavelength of the excitation light radiated from the excitation light source 101 provided in the imaging unit 10 is set according to the photosensitizer to be used as shown in FIG. 4.

Note that, although FIG. 3 show the case where one excitation light source 101 is used, the number of the excitation light source 101 is not limited to one and a plurality of light sources may be prepared according to the kinds of the photosensitizers used in the photodynamic diagnostic device 1. Further, the excitation light source 101 may be configured to cope with a plurality of excitation wavelengths by having a wavelength-conversion mechanism.

No particular limitation is imposed on the type of the excitation light source 101, and various types of publicly known laser light sources may be used by optionally including various lenses and the like for producing diffused light. In such a case, the laser light source may be a continuous wave (CW) laser light source capable of emitting CW laser light or a pulse laser light source capable of emitting pulse laser light. Further, an optical element such as a light emitting diode may be used provided that an output sufficient to excite the photosensitizers is obtained.

The fluorescence imaging device 103 captures an image of the fluorescence from the photosensitizers which are excited by the excitation light from the excitation light source 101 to produce the fluorescence image (i.e., the PDD image). Hereinafter, the fluorescence image produced by the fluorescence imaging device 103 is also referred to as the PDD image. The fluorescence imaging device 103 includes, for example, various imaging elements such as a CCD (Charge Coupled Device) and a CMOS (Complementary Metal Oxide Semiconductor) or a photo detector such as a photomultiplier tube (PMT), and converts a detection result of the fluorescence from the photosensitizers into image data. In this manner, the image data of the fluorescence image derived from the photosensitizers can be produced.

Note that, in the fluorescence imaging device 103 according to the present embodiment, an optical filter 105 that transmits the fluorescence from the photosensitizers but not the excitation light is preferably provided on an upstream side of the fluorescence imaging device 103 to capture an image of the fluorescence from the photosensitizers more clearly. Note that FIG. 3 shows the case where the optical filter 105 is provided outside the fluorescence imaging device 103, however, the optical filter 105 may be provided inside the fluorescence imaging device 103 as long as the optical filter 105 is located on an upstream side of the various imaging elements provided in the fluorescence imaging device 103.

Further, the imaging unit 10 according to the present embodiment preferably further includes an illumination imaging device 107 that captures an image of a part of a human body to which the photosensitizers are administered in advance by utilizing illumination light belonging to a visible light band radiated from the illumination light source 3 to produce an illumination image. In this configuration, it is preferred that a relative positional relation between the illumination imaging device 107 and the fluorescence imaging device 103 (e.g., an angle formed by the optical axes of both imaging devices or the like) is set to a predetermined value in advance, so that, for example, specifying the direction of the optical axis of the illumination imaging device 107 can specify the direction of the optical axis of the fluorescence imaging device 103.

The illumination image produced by the illumination imaging device 107 is captured under the illumination light belonging to a visible light band and thus is an actual image observed by a user of the photodynamic diagnostic device 1 (i.e., the operator of the excision surgery) during the surgery.

Note that the fluorescence imaging device 103 and the illumination imaging device 107 are shown as separate devices in FIG. 3. Regarding this point, the optical filter 105 can be inserted in and removed from the optical axis at a high speed, thus a single imaging device can achieve both functions of the fluorescence imaging device 103 and the illumination imaging device 107 when the imaging device is equipped with an imaging element capable of capturing a color image. However, when the fluorescence imaging device 103 that captures a fluorescence image and the illumination imaging device 107 that captures an illumination image are separately provided as shown in FIG. 3, the stability of the imaging unit 10 can be further improved without the necessity of performing processes such as inserting and removing the optical filter 105.

Further, FIG. 3 shows the case where the illumination light source 3 that radiates the illumination light belonging to a visible light band is provided separately from the imaging unit 10, however, as previously described, the imaging unit 10 itself may include the illumination light source. Such an illumination light source is not particularly limited as long as it can radiate illumination light belonging to a visible light band, and a publicly known light source may be used.

Further, the fluorescence imaging device 103 and the illumination imaging device 107 are shown as separate devices in FIG. 3. However, the fluorescence imaging device 103 and the illumination imaging device 107 can be integrated as shown in FIG. 5. An integrated imaging device (an integrated imaging device 111) shown in FIG. 5 includes, in a camera main body, a fluorescence imaging element 151 where the fluorescence from the lesion part forms an image and an illumination imaging element 153 where the illumination light from the lesion part forms an image. The light from the lesion part is guided to the camera main body through a lens and then divided into two optical paths by a beam splitter BS provided on an optical axis. The optical filter 105 is provided on one optical path and the fluorescence imaging element 151 is provided in the subsequent stage of the optical filter 105. Further, the illumination imaging element 153 is provided on the other optical path.

When the integrated imaging device 111 shown in FIG. 5 is used, unlike the case in FIG. 3, the optical axis of the fluorescence imaging device 103 and the optical axis of the illumination imaging device 107 are aligned with each other, so that the optical axis corresponding to the image formed on the illumination imaging element 153 is aligned in the same direction as the optical axis corresponding to the image formed on the fluorescence imaging element 151. As a result, pre-integration processing prior to integration processing of the fluorescence image and other images, which is described in detail below, can be more easily performed. Further, this configuration can save more space than the one shown in FIG. 3.

In the foregoing, the configuration of the imaging unit 10 according to the present embodiment has been described in detail with reference to FIG. 3 to FIG. 5.

### <Configuration of Arithmetic Processing Unit 20>

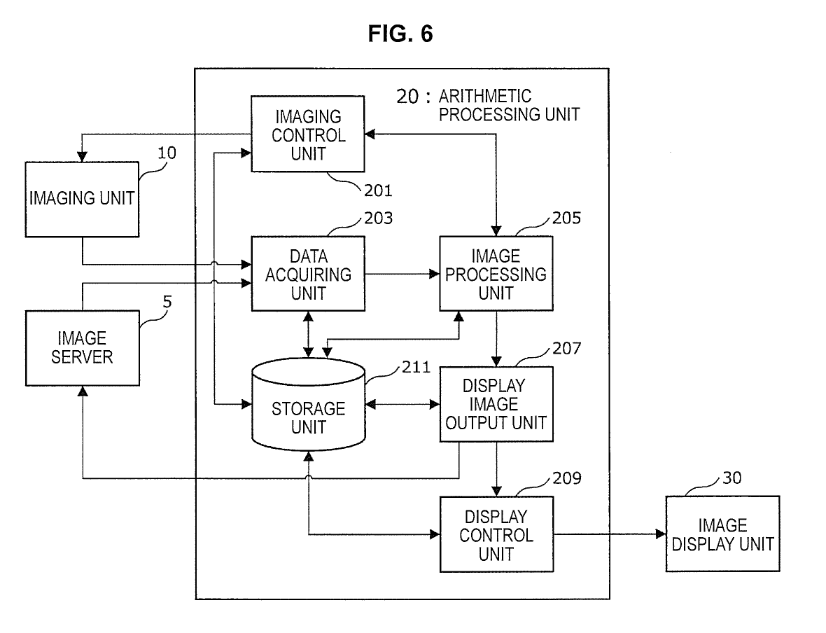

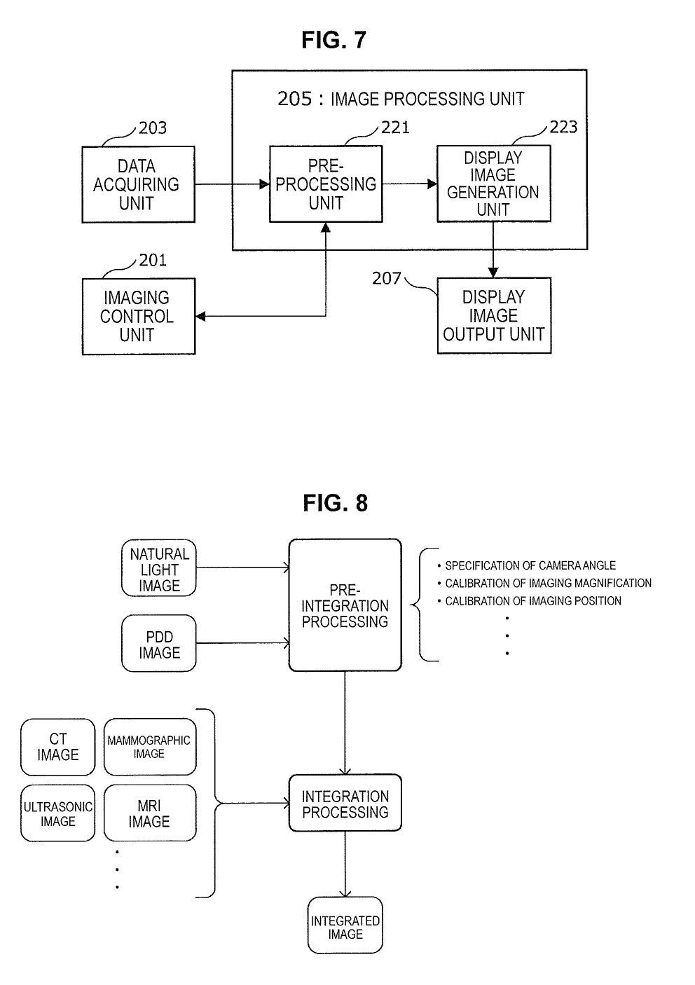

Next, the configuration of the arithmetic processing unit 20 according to the present embodiment will be described in detail with reference to FIG. 6 to FIG. 10. FIG. 6 shows a block diagram schematically illustrating an example of the configurations of the arithmetic processing unit in the photodynamic diagnostic device according to the present embodiment. FIG. 7 shows a block diagram schematically illustrating an example of the configurations of an image processing unit included in the arithmetic processing unit according to the present embodiment. FIG. 8 to FIG. 10 show explanatory diagrams illustrating processes of producing a display image performed in the image processing unit according to the present embodiment.

As schematically shown in FIG. 6, the arithmetic processing unit 20 according to the present embodiment mainly includes an imaging control unit 201, a data acquiring unit 203, an image processing unit 205, a display image output unit 207, a display control unit 209, and a storage unit 211.

The imaging control unit 201 can be achieved, for example, by a CPU (Central Processing Unit), a ROM (Read Only Memory), a RAM (Random Access Memory), a communication device, and the like. The imaging control unit 201 controls various imaging processes in the imaging unit 10. More specifically, the imaging control unit 201 performs on/off control of the excitation light source 101 in the imaging unit 10 and drive control of the fluorescence imaging device 103 and the illumination imaging device 107.

Further, the imaging control unit 201 can perform on/off control of the illumination light in the illumination light source 3. When the imaging unit 10 includes an own illumination light source capable of radiating the illumination light belonging to a visible light band, the imaging control unit 201 can also perform on/off control of the illumination light in such an illumination light source.

The data acquiring unit 203 can be achieved, for example, by a CPU, a ROM, a RAM, a communication device, and the like. The data acquiring unit 203 acquires image data regarding the fluorescence image (the PDD image) and illumination image produced in the imaging unit 10 from the imaging unit 10 as appropriate. Further, the data acquiring unit 203 acquires image data regarding the various diagnostic images stored in an external server such as an image server 5 from the pertinent external server at any time as needed. The data acquiring unit 203 outputs the acquired image data to the image processing unit 205 described below. Further, the data acquiring unit 203 may store the acquired image data in the storage unit 211 or the like described below.

The image processing unit 205 can be achieved, for example, by a CPU, a ROM, a RAM, and the like. The image processing unit 205 applies image processing described below in detail to the fluorescence image (the PDD image) outputted from the data acquiring unit 203 to produce an integrated image in which a first image representing the positional relation of at least a part of a human body is integrated into the fluorescence image. The integrated image may be a two-dimensional image or a three-dimensional image which can be stereoscopically viewed. In this configuration, the first image integrated into the fluorescence image represents the positional relation of at least a part of a human body and is at least one of the image capturing the operation field during the excision surgery of the malignant tumors into which the photosensitizers are incorporated (i.e., the illumination image) or the diagnostic image representing the location of the malignant tumors (i.e., the various diagnostic images stored in the image server 5 or the like).

Integrating the image representing the positional relation of at least a part of a human body such as the illumination image into the fluorescence image allows a user of the photodynamic diagnostic device 1 to easily recognize the presence of the malignant tumors and, if present, easily recognize the location of the malignant tumors in the operation field. This results in a reduction in the risk of positive surgical margins described above. Further, when such an integrated image is integrated with the various diagnostic images representing the positional relation of the malignant tumors in addition to the positional relation of at least a part of a human body, such as a mammographic image, a CT image, an MRI image, and an ultrasonic image, the integrated image can be further superimposed with information on how the malignant tumors have spread, suggested from the diagnostic images.

Note that the image processing unit 205 preferably applies various preprocesses to the fluorescence image before integrating the first image described above into the fluorescence image in the production of the integrated image. If it is preferable to change imaging conditions of the fluorescence image when performing the preprocesses, the image processing unit 205 can change the imaging conditions of the fluorescence image cooperatively with the imaging control unit 201.

The image processing unit 205 may further superimpose, on the produced integrated image, various display objects that emphasize a region where a fluorescence image corresponding to the location of the malignant tumors is formed (a fluorescence image forming region). Further, the image processing unit 205 may change a color tone of the fluorescence image forming region to a color tone different from the original fluorescent color (e.g., colors that does not exist in a living body, such as a pink color and a green color) to emphasize the presence and location of the fluorescence image forming region.

Detailed configuration of the image processing unit 205 performing such production processing of the integrated image will be described again below.

The image processing unit 205 outputs image data regarding the produced integrated image to a display image output unit 207 described below.

The display image output unit 207 can be achieved, for example, by a CPU, a ROM, a RAM, a communication device, and the like. The display image output unit 207 outputs the integrated image produced in the image processing unit 205 by integrating the first image different from the PDD image into the fluorescence image (the PDD image), to the outside of the arithmetic processing unit 20. When such an integrated image is outputted to a display or the like provided as an image display unit 30, the image data of such an integrated image is outputted to a display control unit 209 to cause the display control unit 209 to perform display control of the integrated image.

Further, the display image output unit 207 may output the image data of the produced integrated image and the image data of the PDD image as a source of the integrated image to an external server such as the image server 5. Further, the display image output unit 207 may output the produced integrated image as a print.

The display control unit 209 can be achieved, for example, by a CPU, a ROM, a RAM, a communication device, and the like. The display control unit 209 performs display control of the integrated image, which is obtained by integrating he first image different from the PDD image into the fluorescence image (the PDD image) and transmitted from the display image output unit 207, when the integrated image is displayed on an output device such as a display provided in the image display unit 30, an output device provided outside the photodynamic diagnostic device 1, or the like. This allows a user of the photodynamic diagnostic device 1 to instantly recognize the produced integrated image.

The storage unit 211 can be achieved, for example, by the RAM, the storage device, and the like, provided in the arithmetic processing unit 20 according to the present embodiment. The storage unit 211 appropriately records various parameters, the progress of processing, and the like, which are needed to be stored when the arithmetic processing unit 20 according to the present embodiment performs certain processing, or various databases, programs, and the like. The storage unit 211 can be freely accessed from the imaging control unit 201, the data acquiring unit 203, the image processing unit 205, the display image output unit 207, the display control unit 209, and the like to read and write data.

### [Configuration of Image Processing Unit 205]

Next, the configuration of the image processing unit 205 will be described in detail with reference to FIG. 7 to FIG. 10.

As schematically shown in FIG. 7, the image processing unit 205 according to the present embodiment includes a pre-processing unit 221 and a display image generation unit 223.

The pre-processing unit 221 can be achieved, for example, by a CPU, a ROM, a RAM, and the like. The pre-processing unit 221 performs pre-integration processing of the fluorescence image (the PDD image) and illumination image transmitted from the data acquiring unit 203, which includes at least processing for adjusting display magnification and processing for positioning with the first image described above.

As shown in FIG. 8, the pre-integration processing preferably includes at least processing for specifying camera angle, processing for calibrating imaging magnification, and processing for calibrating imaging position.

The processing for specifying camera angle specifies the direction of a camera by recognizing at least a part of a human body of the illumination image by using, for example, publicly known image recognition processing or the like. This makes it possible to determine the direction of the optical axis of the illumination imaging device 107, for example, whether it faces the cranial or caudal end of the human body. In addition, further detailed recognition processing makes it possible to determine the specific direction of the optical axis of the illumination imaging device 107 (a rotation angle from a certain reference direction).

In this configuration, the relative positional relation is preset between the illumination imaging device 107 and the fluorescence imaging device 103, thus performing the above-mentioned processing using the illumination image makes it possible to determine the direction of the optical axis of the fluorescence imaging device 103.

The processing for specifying camera angle only needs to be performed at least once as long as the imaging processing is performed under the same imaging conditions in the fluorescence imaging device 103 and the illumination imaging device 107. Further, when the imaging conditions of the fluorescence imaging device 103 and illumination imaging device 107 are changed, the processing for specifying camera angle is performed each time.

The processing for calibrating imaging magnification calibrates an imaging magnification of a camera at a focus position (i.e., a patient in the operation field) of the imaging device. Determining the range of the focus position of the illumination imaging device 107 to be included in the viewing field enables to determine a difference in imaging magnifications between the first image (different from the illumination image) to be integrated and the illumination image. This makes it possible to recognize what extent a captured image needs to be scaled up (or down) in integrating the illumination image and the first image. Further, the relative positional relation is known between the illumination imaging device 107 and the fluorescence imaging device 103, thus determining the degree of calibration of the imaging magnification of the illumination image enables to determine the degree of calibration of the imaging magnification of the fluorescence image. Note that, when zooming is performed in the imaging device after the magnification is calibrated as described above, the calibration is appropriately performed on the basis of the zooming magnification.

The processing for calibrating imaging position calibrates an imaging position so as to match the positional relation between the fluorescence image and the first image. More specifically, positioning parameters for aligning a position of a specific organ of a human body (e.g., a nipple or the like in the surgery of the breast cancer) in the illumination image and a position of the same organ in the first image are calculated by using knowledge on the imaging direction and the display magnification. Then, the positions of the fluorescence image and the first image are aligned by using the calculated positioning parameters. During this process, when the specific organ of a human body included in the first image is not included in the viewing field of the illumination image, the pre-processing unit 221 changes the imaging conditions to include the organ of interest in the viewing field cooperatively with imaging control unit 201.

Performing the pre-integration processing as described above enables to integrate the first image (various diagnostic images in particular), which is usually displayed larger than its actual size, with the fluorescence image and illumination image obtained during the surgery after matching their image magnifications.

When the fluorescence image and the first image are displayed in the same magnification, the operator can precisely compare the diagnostic image with the PDD image and an observation image of the operation field obtained during the surgery. As a result, the operator can easily recognize the location of the malignant tumors such as cancer shown in the diagnostic image on the basis of the positional relation of a human body and easily determine a region to be excised.

In this process, the imaging unit 10 according to the present embodiment preferably adopts the integrated imaging device 111 shown in FIG. 5 to more easily perform the various calibration processes described above.

Note that, in the above description, the pre-processing unit 221 applies the various pre-integration processes described above mainly to the fluorescence image, however, the pre-processing unit 221 may apply the same pre-integration processes to the illumination image. Further, the pre-processing unit 221 may apply various image processes, such as enlargement, reduction, and rotation of images, also to the first image (e.g., the various diagnostic images) other than the illumination image to be integrated.

After applying the pre-integration processing to the target captured images as described above, the pre-processing unit 221 outputs image data after the pre-integration processing to the display image generation unit 223.

The display image generation unit 223 can be achieved, for example, by a CPU, a ROM, a RAM, and the like. The display image generation unit 223 produces an integrated image into which the fluorescence image and the first image representing the positional relation of at least a part of a human body are integrated by using the image data after the pre-integration processing, which are transmitted from the pre-processing unit 221. As schematically shown in FIG. 8, this makes it possible to produce an integrated image into which the fluorescence image and illumination image after the pre-integration processing are integrated, an integrated image into which the fluorescence image after the pre-integration processing and at least one of the diagnostic images such as a mammographic image, a CT image, an MRI image, and an ultrasonic image are integrated, an integrated image into which the fluorescence image and illumination image after the pre-integration processing, and at least one of the diagnostic images are integrated, and the like.

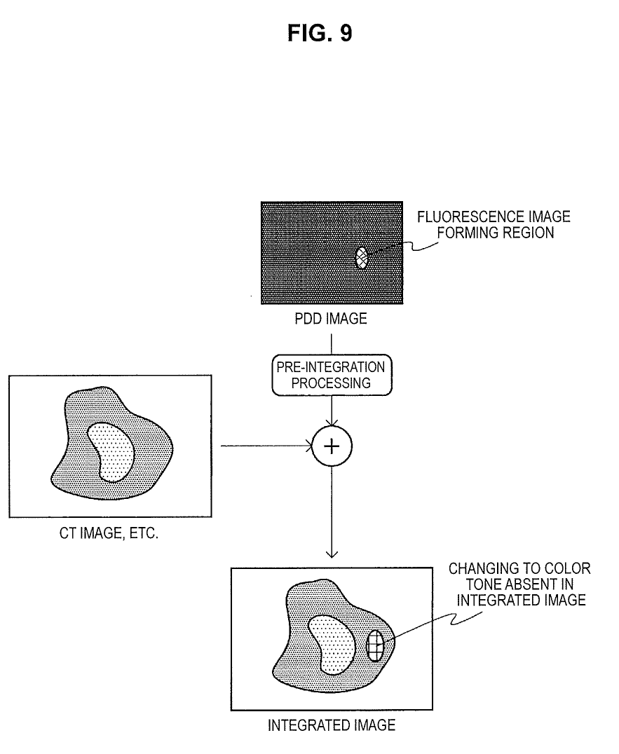

During this process, as schematically shown in FIG. 9, the display image generation unit 223 preferably changes the color tone of the fluorescence image forming region of the fluorescence image (the PDD image) from the original fluorescent color tone derived from the photosensitizers to a color tone which does not exist in the integrated image. This can prevent a user of the photodynamic diagnostic device 1 referring to the integrated image from overlooking the presence of the fluorescence image forming region, which is otherwise buried in the integrated image, and reduce the risk of positive surgical margins.

Examples of a method of changing the color tone may include the one in which image luminance information obtained from the fluorescence image (the PDD image) is inputted into a green (G) channel of the image data of the integrated image. Further, image data regarding the color tone of the fluorescence image forming region of the fluorescence image may be directly rewritten and changed into a value corresponding to a desired color tone.

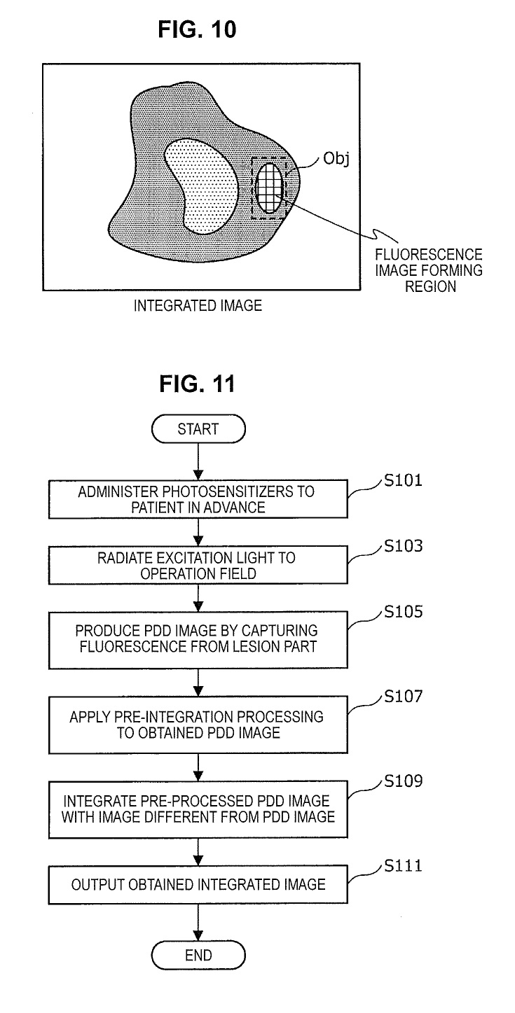

Further, the display image generation unit 223 may further superimpose a display object obj that emphasizes the fluorescence image forming region on the integrated image to emphatically display the fluorescence image forming region. Examples of such a display object obj includes the one surrounding the fluorescence image forming region, for example, with a dotted line as schematically shown in FIG. 10. Further, various marker objects showing the fluorescence image forming region may be superimposed on the integrated image, and a display effect, such as displaying the fluorescence image forming region by blinking, may be used in combination. Through the further superimposition of the display object obj, a user of the photodynamic diagnostic device 1 becomes less likely to overlook the presence of the fluorescence image forming region, thereby enabling to lower the risk of positive surgical margins.

The display image generation unit 223 outputs the image data regarding the integrated image thus produced to the display image output unit 207. This allows a user of the photodynamic diagnostic device 1 to perform PDD using various methods including image display on the image display unit 30.

For example, in the recent surgery of breast cancer, an excision target region is commonly marked like the breast of the patient by a marker pen on the basis of the diagnostic image such as, for example, a mammography image, as described above. However, such a process converts the three-dimensional excision region for the surgery of the diagnostic image to perspective plan view information, thereby eliminating a part of information held in the diagnostic image. On the other hand, using the integrated image described above makes it possible to utilize the information held in the diagnostic image more efficiently and compensate the information that may have been lost in a conventional method.

In the foregoing, the configuration of the image processing unit 205 according to the present embodiment has been described in detail with reference to FIG. 7.

Examples of functions of the arithmetic processing unit 20 according to the present embodiment have been described above. The respective constituent elements described above may be configured using universal members and circuits, or may be configured using hardware specialized for the functions of the constituent elements. Further, all of the functions of the constituent elements may be fulfilled by a CPU and the like. Thus, a configuration to be used can be appropriately changed in accordance with a technical level of any occasion at which the present embodiment is implemented.

Note that a computer program for achieving each function of the arithmetic processing unit according to the present embodiment described above can be produced and installed in a personal computer and the like. Further, a computer-readable recording medium on which the computer program is stored can also be provided. The recording medium is, for example, a magnetic disk, an optical disc, a magneto-optical disc, a flash memory, or the like. Furthermore, the computer program described above may be distributed through, for example, a network, without using the recording medium.

### <Photodynamic Diagnostic Method>

Next, processes of a photodynamic diagnostic method according to the present embodiment will be briefly described with reference to FIG. 11. FIG. 11 shows a flowchart illustrating an example of the processes of the photodynamic diagnostic method according to the present embodiment.

In the photodynamic diagnostic method according to the present embodiment, a patient is first administered with specific photosensitizers in advance (Step S101) to accumulate the photosensitizers in malignant tumors such as cancer. Next, during the surgery, the imaging unit 10 is driven under the control of the arithmetic processing unit 20 in the photodynamic diagnostic device 1 to radiate the excitation light having a wavelength capable of exciting the photosensitizers to an operation field that includes a region where the malignant tumors likely exist (a lesion part) from the excitation light source 101 of the imaging unit 10 in the photodynamic diagnostic device 1 (Step S103).

When the photosensitizers are accumulated in the operation field of interest, fluorescence is generated by the radiated excitation light. Then, the fluorescence from the lesion part is captured by the fluorescence imaging device 103 of the imaging unit 10 in the photodynamic diagnostic device 1 to produce a PDD image. Further, it is preferable that an illumination image is also produced using the illumination imaging device 107 of the imaging unit 10 in addition to the production of the PDD image.

After various images are produced by the imaging unit 10, image data of the produced images are outputted to the arithmetic processing unit 20. The data acquiring unit 203 of the arithmetic processing unit 20 acquires the image data of the various images produced by the imaging unit 10 and outputs the acquired image data to the pre-processing unit 221 of the image processing unit 205.

The pre-processing unit 221 of the image processing unit 205 applies the pre-integration processing described above to the PDD image and the illumination image (Step S107). Then, the pre-processing unit 221 outputs image data of the PDD image and illumination image after the pre-integration processing to the display image generation unit 223.

Subsequently, the display image generation unit 223 of the image processing unit 205 integrates the PDD image and an image different from the PDD image by the above-mentioned method using the diagnostic image and the like separately acquired from the image server 5 or the like by the data acquiring unit 203 (Step S109). In this manner, an integrated image according to the present embodiment is produced. The display image generation unit 223 then outputs image data regarding the integrated image thus produced to the display image output unit 207.

The display image output unit 207 outputs the image data regarding the integrated image outputted from the image processing unit 205 (Step S111) For example, when the integrated image is displayed on a display or the like of the image display unit 30, the display image output unit 207 outputs the image data regarding the integrated image to the display control unit 209 to cause the display control unit 209 to perform display control of the image display unit 30. In this manner, the produced integrated image is presented to a user of the photodynamic diagnostic device 1.

In the foregoing, one example of the processes of the photodynamic diagnostic method according to the present embodiment has been briefly described with reference to FIG. 11.

### (Hardware Configuration)

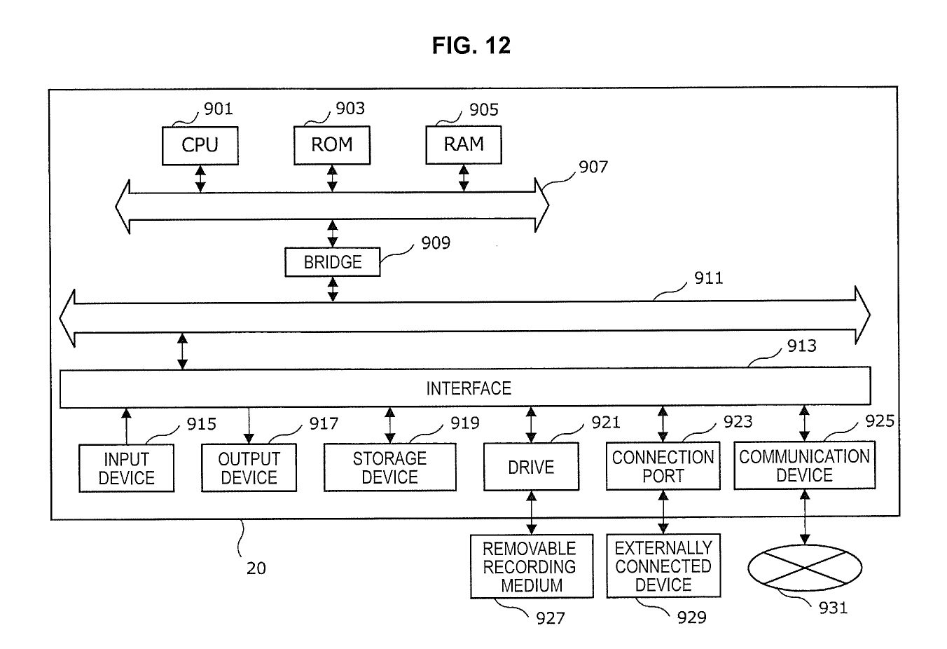

Next, the hardware configuration of the arithmetic processing unit 20 according to the embodiment of the present disclosure will be described in detail with reference to FIG. 12. FIG. 12 is a block diagram for illustrating the hardware configuration of the arithmetic processing unit 20 according to the embodiment of the present disclosure.

The arithmetic processing unit 20 mainly includes a CPU 901, a ROM 903, and a RAM 905. Furthermore, the arithmetic processing device 20 also includes a host bus 907, a bridge 909, an external bus 911, an interface 913, an input device 915, an output device 917, a storage device 919, a drive 921, a connection port 923, and a communication device 925.

The CPU 901 serves as an arithmetic processing device and a control device, and controls the overall operation or a part of the operation of the arithmetic processing unit 20 according to various programs recorded in the ROM 903, the RAM 905, the storage device 919, or a removable recording medium 927. The ROM 903 stores programs, operation parameters, and the like used by the CPU 901. The RAM 905 primarily stores programs used in execution of the CPU 901 and parameters and the like varying as appropriate during the execution. These are connected with each other via the host bus 907 configured from an internal bus such as a CPU bus or the like.

The host bus 907 is connected to the external bus 911 such as a PCI (Peripheral Component Interconnect/Interface) bus via the bridge 909.

The input device 915 is an operation means operated by a user, such as a mouse, a keyboard, a touch panel, buttons, a switch and a lever. Also, the input device 915 may be a remote control means (a so-called remote control) using, for example, infrared light or other radio waves, or may be an externally connected device 929 such as a mobile phone or a PDA conforming to the operation of the photodynamic diagnostic device 1. Furthermore, the input device 915 is configured from, for example, an input control circuit that generates an input signal on the basis of information inputted by a user using the operation means described above and outputs the input signal to the CPU 901. The user can input various data to the photodynamic diagnostic device 1 and can instruct the photodynamic diagnostic device 1 to perform processing by operating this input device 915.

The output device 917 is configured from a device capable of visually or audibly notifying acquired information to a user. Examples of such device include display devices such as a CRT display device, a liquid crystal display device, a plasma display device, an EL display device and lamps, audio output devices such as a speaker and a headphone, a printer, a mobile phone, a facsimile machine, and the like. For example, the output device 917 outputs a result obtained by various processings performed by the arithmetic processing unit 20. More specifically, the display device displays, in the form of texts or images, a result obtained by various processes performed by the arithmetic processing unit 20. On the other hand, the audio output device converts an audio signal such as reproduced audio data and sound data into an analog signal, and outputs the analog signal.

The storage device 919 is a device for storing data configured as an example of a storage unit of the arithmetic processing unit 20 and is used to store data. The storage device 919 is configured from, for example, a magnetic storage device such as a HDD (Hard Disk Drive), a semiconductor storage device, an optical storage device, or a magneto-optical storage device. This storage device 919 stores programs to be executed by the CPU 901, various data, and various data obtained externally, or the like.

The drive 921 is a reader/writer for recording medium, and is embedded in the arithmetic processing unit 20 or attached externally thereto. The drive 921 reads information recorded in the attached removable recording medium 927 such as a magnetic disk, an optical disk, a magneto-optical disk, or a semiconductor memory, and outputs the read information to the RAM 905. Furthermore, the drive 921 can write in the attached removable recording medium 927 such as a magnetic disk, an optical disk, a magneto-optical disk, or a semiconductor memory. The removable recording medium 927 is, for example, a DVD medium, an HD-DVD medium, or a Blu-ray (registered trademark) medium. The removable recording medium 927 may be a CompactFlash (CF; registered trademark), a flash memory, an SD memory card (Secure Digital Memory Card), or the like. Alternatively, the removable recording medium 927 may be, for example, an IC card (Integrated Circuit Card) equipped with a non-contact IC chip or an electronic appliance.

The connection port 923 is a port for allowing devices to directly connect to the arithmetic processing unit 20. Examples of the connection port 923 include a USB (Universal Serial Bus) port, an IEEE1394 port, a SCSI (Small Computer System Interface) port, and the like. Other examples of the connection port 923 include an RS-232C port, an optical audio terminal, an HDMI (High-Definition Multimedia Interface) port, and the like. By the externally connected device 929 connecting to this connection port 923, the photodynamic diagnostic device 1 directly obtains various data from the externally connected device 929 and provides various data to the externally connected device 929.

The communication device 925 is a communication interface configured from, for example, a communication device for connecting to a communication network 931. The communication device 925 is, for example, a wired or wireless LAN (Local Area Network), Bluetooth (registered trademark), a communication card for WUSB (Wireless USB), or the like. Alternatively, the communication device 925 may be a router for optical communication, a router for ADSL (Asymmetric Digital Subscriber Line), a modem for various communications, or the like. This communication device 925 can transmit and receive signals and the like in accordance with a predetermined protocol such as TCP/IP on the Internet and with other communication devices, for example. The communication network 931 connected to the communication device 925 is configured from a network and the like, which is connected via wire or wirelessly, and may be, for example, the Internet, a home LAN, infrared communication, radio wave communication, satellite communication, or the like.

Heretofore, an example of the hardware configuration capable of realizing the functions of the arithmetic processing unit 20 according to the embodiment of the present disclosure has been shown. Each of the structural elements described above may be configured using a general-purpose material, or may be configured from hardware dedicated to the function of each structural element. Accordingly, the hardware configuration to be used can be changed as appropriate according to the technical level at the time of carrying out the present embodiment.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A photodynamic diagnostic device including:

an imaging unit including an excitation light source that radiates excitation light having a specific wavelength and a fluorescence imaging device that captures an image of fluorescence from a photosensitizer excited by the excitation light to produce a fluorescence image; and

an arithmetic processing unit including an image processing unit that applies predetermined image processing to the fluorescence image,

in which the image processing unit integrates a first image representing a positional relation of at least a part of a human body into the fluorescence image to produce an integrated image.

(2)

The photodynamic diagnostic device according to (1), in which the image processing unit applies, to the fluorescence image, pre-integration processing that includes at least a process of adjusting display magnification and a process of aligning position with the first image, and then integrates the fluorescence image after the pre-integration processing with the first image.

(3)

The photodynamic diagnostic device according to (2), in which

the imaging unit further includes an illumination imaging device that captures an image of a part of a human body to which the photosensitizer is administered in advance by utilizing illumination light belonging to a visible light band to produce an illumination image, in which a relative positional relation between the illumination imaging device and the fluorescence imaging device is preset, and

the image processing unit performs

specifying imaging directions of the illumination imaging device and the fluorescence imaging device by recognizing at least a part of a human body of the illumination image,

adjusting display magnifications of the illumination image and the fluorescence image to be produced on the basis of imaging magnifications of the illumination imaging device and the fluorescence imaging device,

calculating a positioning parameter for aligning a position of an organ of a human body in the illumination image and a position of the organ of the human body in the first image by utilizing the imaging directions and the display magnifications, and

aligning positions of the fluorescence image and the first image by utilizing the calculated positioning parameter.

(4)

The photodynamic diagnostic device according to (3), in which the fluorescence imaging device and the illumination imaging device are integrated, and an integrated imaging device divides incident light into two optical paths to produce the fluorescence image and the illumination image.

(5)

The photodynamic diagnostic device according to (3) or (4), in which

the imaging unit further includes an illumination light source that radiates the illumination light belonging to a visible light band,

the arithmetic processing unit further includes an imaging control unit that controls the imaging processing in the imaging unit, and

the imaging control unit performs on/off control of the excitation light source and the illumination light source and drive control of the fluorescence imaging device and the illumination imaging device.

(6)

The photodynamic diagnostic device according to any one of (1) to (5), in which the image processing unit changes a color tone of a region of the integrated image corresponding to a fluorescence image forming region of the fluorescence image to a color tone that does not exist in the first image.

(7)

The photodynamic diagnostic device according to any one of (1) to (6), in which the image processing unit further superimposes, on the integrated image, a display object that emphasizes the fluorescence image forming region of the integrated image.

(8)

The photodynamic diagnostic device according to any one of (1) to (7), in which the first image is at least one of an image capturing an operation field of an excision surgery of a malignant tumor into which the photosensitizer is incorporated, or a diagnostic image indicating a location of the malignant tumor.

(9)

The photodynamic diagnostic device according to (8), in which the diagnostic image is at least one of a fluoroscopic image or a sectional image of at least a part of a human body.

(10)

The photodynamic diagnostic device according to (9), in which the fluoroscopic image or the sectional image is a mammographic image, a CT image, an MRI image, or an ultrasonic image.

(11)

The photodynamic diagnostic device according to any one of (1) to (10), in which the arithmetic processing unit acquires the first image from an externally provided image server and integrates the first image with the fluorescence image.

(12)

A photodynamic diagnostic method including:

producing a fluorescence image by radiating excitation light having a specific wavelength from an excitation light source and capturing an image of fluorescence from a photosensitizer excited by the excitation light by a fluorescence imaging device; and

producing an integrated image by integrating a first image representing a positional relation of at least a part of a human body into the produced fluorescence image.

### REFERENCE SIGNS LIST

1 photodynamic diagnostic device
3 illumination light source
5 image server
10 imaging unit
20 arithmetic processing unit
30 image display unit
101 excitation light source
103 fluorescence imaging device
105 optical filter
107 illumination imaging device
111 integrated imaging device
151 fluorescence imaging element
153 illumination imaging element
201 imaging control unit
203 data acquiring unit
205 image processing unit
207 display image output unit
209 display control unit
211 storage unit
221 pre-processing unit
223 display image generation unit

1. A photodynamic diagnostic device comprising:
an imaging unit including an excitation light source that radiates excitation light having a specific wavelength and a fluorescence imaging device that captures an image of fluorescence from a photosensitizer excited by the excitation light to produce a fluorescence image; and
an arithmetic processing unit including an image processing unit that applies predetermined image processing to the fluorescence image,
wherein the image processing unit integrates a first image representing a positional relation of at least a part of a human body into the fluorescence image to produce an integrated image.

2. The photodynamic diagnostic device according to claim 1, wherein the image processing unit applies, to the fluorescence image, pre-integration processing that includes at least a process of adjusting display magnification and a process of aligning position with the first image, and then integrates the fluorescence image after the pre-integration processing with the first image.

3. The photodynamic diagnostic device according to claim 2, wherein
the imaging unit further includes an illumination imaging device that captures an image of a part of a human body to which the photosensitizer is administered in advance by utilizing illumination light belonging to a visible light band to produce an illumination image, wherein a relative positional relation between the illumination imaging device and the fluorescence imaging device is preset, and
the image processing unit performs
specifying imaging directions of the illumination imaging device and the fluorescence imaging device by recognizing at least a part of a human body of the illumination image,
adjusting display magnifications of the illumination image and the fluorescence image to be produced on the basis of imaging magnifications of the illumination imaging device and the fluorescence imaging device,
calculating a positioning parameter for aligning a position of an organ of a human body in the illumination image and a position of the organ of the human body in the first image by utilizing the imaging directions and the display magnifications, and
aligning positions of the fluorescence image and the first image by utilizing the calculated positioning parameter.

4. The photodynamic diagnostic device according to claim 3, wherein the fluorescence imaging device and the illumination imaging device are integrated, and an integrated imaging device divides incident light into two optical paths to produce the fluorescence image and the illumination image.

5. The photodynamic diagnostic device according to claim 3, wherein
the imaging unit further includes an illumination light source that radiates the illumination light belonging to a visible light band,
the arithmetic processing unit further includes an imaging control unit that controls the imaging processing in the imaging unit, and
the imaging control unit performs on/off control of the excitation light source and the illumination light source and drive control of the fluorescence imaging device and the illumination imaging device.

6. The photodynamic diagnostic device according to claim 1, wherein the image processing unit changes a color tone of a region of the integrated image corresponding to a fluorescence image forming region of the fluorescence image to a color tone that does not exist in the first image.

7. The photodynamic diagnostic device according to claim 1, wherein the image processing unit further superimposes, on the integrated image, a display object that emphasizes the fluorescence image forming region of the integrated image.

8. The photodynamic diagnostic device according to claim 1, wherein the first image is at least one of an image capturing an operation field of an excision surgery of a malignant tumor into which the photosensitizer is incorporated, or a diagnostic image indicating a location of the malignant tumor.

9. The photodynamic diagnostic device according to claim 8, wherein the diagnostic image is at least one of a fluoroscopic image or a sectional image of at least a part of a human body.

10. The photodynamic diagnostic device according to claim 9, wherein the fluoroscopic image or the sectional image is a mammographic image, a CT image, an MRI image, or an ultrasonic image.

11. The photodynamic diagnostic device according to claim 1, wherein the arithmetic processing unit acquires the first image from an externally provided image server and integrates the first image with the fluorescence image.

12. A photodynamic diagnostic method comprising:
producing a fluorescence image by radiating excitation light having a specific wavelength from an excitation light source and capturing an image of fluorescence from a photosensitizer excited by the excitation light by a fluorescence imaging device; and
producing an integrated image by integrating a first image representing a positional relation of at least a part of a human body into the produced fluorescence image.
